(19) United States
(12) Patent Application Publication
(10) Pub. No.: US 2008/0288010 A1
(43) Pub. Date: Nov. 20, 2008

(54) Subcutaneous diaphragm stimulation device and method for use

(75) Inventors: Amir J. Tehrani, Los Altos, CA (US); Rose Province, San Jose, CA (US)

Correspondence Address: LEVINE BAGADE HAN LLP, 2483 EAST BAYSHORE ROAD, SUITE 100, PALO ALTO, CA, 94303, US

(73) Assignee: RMX, LLC, San Francisco, CA (US)

(21) Appl. No.: 12/150,045

(22) Filed: Apr. 23, 2008

Related U.S. Application Data
(63) Continuation-in-part of application No. 12/082,057, filed on Apr. 8, 2008.
Application No. 12/082,057 is a continuation-in-part of application No. 12/069,823, filed on Feb. 13, 2008.
Application No. 12/069,823 is a continuation-in-part of application No. 12/004,932, filed on Dec. 21, 2007.
Application No. 12/004,932 is a continuation-in-part of application No. 11/981,342, filed on Oct. 31, 2007.
Application No. 11/981,342 is a continuation-in-part of application No. 11/480,074, filed on Jun. 29, 2006.
Application No. 11/480,074 is a continuation-in-part of application No. 11/271,726, filed on Nov. 10, 2005.
Application No. 11/271,726 is a continuation-in-part of application No. 10/966,484, filed on Oct. 15, 2004.
Application No. 10/966,484 is a continuation-in-part of application No. 10/966,474, filed on Oct. 15, 2004.
Application No. 10/966,474 is a continuation-in-part of application No. 10/966,421, filed on Oct. 15, 2004.
Application No. 10/966,421 is a continuation-in-part of application No. 10/966,472, filed on Oct. 15, 2004.
Application No. 10/966,472 is a continuation-in-part of application No. 10/686,891, filed on Oct. 15, 2003.
(60) Provisional application No. 60/931,171, filed on May 22, 2007.

(51) Int. Cl. A61N 1/39

(52) U.S. Cl. 607/5; 607/42

(57) ABSTRACT

A cardiovascular treatment assembly is provided including a cardiac rhythm management device and an electrical stimulation device configured to elicit a diaphragm contraction to thereby treat heart failure.

### RELATED APPLICATION DATA

This application claims priority of Provisional Application No. 60/931,171 and; and is a continuation in part of U.S. application Ser. No. 12/082,057 entitled: DEVICE AND METHOD FOR TREATING CARDIOVASCULAR DISORDERS OF THE CARDIOVASCULAR SYSTEM OR HEART filed Apr. 8, 2008; and is a continuation in part U.S. application Ser. No. 12/069,823 filed Feb. 13, 2008, and of U.S. application Ser. No. 12/004,932 filed Dec. 21, 2007; and of U.S. application Ser. No. 11/981,342 filed Oct. 31, 2007; and of U.S. application Ser. No. 11/480,074 filed Jun. 29, 2006 and of U.S. application Ser. No. 11/271,315 filed Nov. 10, 2005; and of U.S. application Ser. No. 11/271,554 filed Nov. 10, 2005; and of U.S. application Ser. No. 11/271,353 filed Nov. 10, 2005; and of U.S. application Ser. No. 11/271,264 filed Nov. 10, 2005; and of U.S. patent application Ser. No. 10/966,487 filed Oct. 15, 2004; and of U.S. application Ser. No. 11/480,074 filed Jun. 29, 2006 which is a continuation in part of U.S. application Ser. No. 11/271,726 filed Nov. 10, 2005 which is a continuation in part of U.S. application Ser. No. 10/966,484 filed Oct. 15, 2004; U.S. application Ser. No. 10/966,474, filed Oct. 15, 2004; U.S. application Ser. No. 10/966,421, filed Oct. 15, 2004; and U.S. application Ser. No. 10/966,472 filed Oct. 15, 2004 which are continuations in part of U.S. application Ser. No. 10/686,891 filed Oct. 15, 2003 entitled: BREATHING DISORDER DETECTION AND THERAPY DELIVERY DEVICE AND METHOD all of which are incorporated in their entirety herein by reference without limitation.

### FIELD OF THE INVENTION

The present invention relates to a system and method for providing diaphragm stimulation with a subcutaneously implanted device. The present invention further relates to a defibrillator used in combination with a diaphragm stimulation device that may be used to treat cardiovascular heart failure (or dysfunction) patients.

### BACKGROUND OF THE INVENTION

Diaphragm stimulation using various stimulation devices has been proposed in a number of configurations. Phrenic nerve stimulation has been proposed using nerve cuffs or transvenously implanted leads. Stimulation electrodes have been implanted on a diaphragm. These devices have required more invasive surgical procedures. Transcutaneous phrenic nerve stimulation electrodes have been used experimentally but may not stay in place or be convenient for a subject for a longer term therapeutic use. Accordingly it would be desirable to provide alternative phrenic nerve/diaphragm stimulation devices.

In copending provisional Application entitled DEVICE AND METHOD FOR TREATING DISORDERS OF THE CARDIOVASCULAR SYSTEM OR HEART, filed on even date herewith and incorporated entirely herein by reference without limitation, electrical stimulation is described to elicit a diaphragm response to provide therapy to patients with heart failure, heart dysfunction or other cardiovascular related disease. Such stimulation may be provided to treat heart failure/dysfunction or other cardiovascular disease and/or may be provided to treat disordered breathing that frequently is associated with heart failure. Such patients may be using a cardiac rhythm management device such as a pacemaker, CRT or ICD or may at some time be a candidate for such device. Accordingly, it would be desirable to provide a device that provides stimulation to elicit a diaphragm response that may work in concert with one or more such devices.

A relatively large population of heart failure patients has some risk of ventricular arrhythmia. Subcutaneous defibrillators have been proposed for class II and III heart failure patients that represent a larger group of heart failure patients than a traditional ICD group of patients. While these defibrillators are not currently designed to provide the more complex stimulation and detection algorithms available in a classic ICD device, they serve a greater patient population at risk of sudden cardiac death. The subcutaneous defibrillator provides an option for a more basic life saving treatment at a lower cost and serving a great patient population. A substantial portion of ventricular arrhythmia patients also have some form of sleep disordered breathing, particularly central and/or obstructive sleep apneas. It would be desirable to provide an implantable device(s) and/or a device(s) with a lower risk and/or cost associated with implantation that may treat ventricular arrhythmias as well as sleep disordered breathing. It would also be desirable to provide an implantable device and/or a device with a lower risk and/or cost associated with implantation that may treat ventricular arrhythmias as well as one or more aspects or conditions associated with heart failure.

Some sleep disordered breathing patients and especially obstructive sleep apnea patients have a prevalence of arrhythmias and sudden cardiac deaths. Accordingly, it would be desirable to provide a device and method to treat these patients.

### SUMMARY OF THE INVENTION

In accordance with one aspect of the invention, a subcutaneously implanted diaphragm stimulation device is provided to treat one or more diseases, disorders and conditions that may relate to, have co-morbidities with, affect, be affected by respiratory or lung health status, respiration, ventilation, or blood gas levels. Such diseases and disorders may include but are not limited to obstructive respiratory disorders, restrictive respiratory disorders, vascular respiratory disorders, upper airway resistance syndrome, snoring, obstructive apnea; central respiratory disorders, central apnea; hypopnea, hypoventilation, obesity hypoventilation syndrome other respiratory insufficiencies, inadequate ventilation or gas exchange, chronic obstructive pulmonary diseases; asthma; emphysema; chronic bronchitis; circulatory disorders; hemodynamic disorders; hypertension; heart disease; chronic heart failure; cardiac rhythm disorders; neurodegenerative disorders, ALS, MS, obesity or injuries in particular affecting breathing or ventilation.

In accordance with another aspect of the invention stimulation is provided to reduce breathing disorders to thereby improve condition of a heart failure patient.

In accordance with one aspect of the invention, stimulation is provided to the diaphragm or phrenic nerve to elicit a diaphragm response to thereby provide a therapeutic effect for a heart failure or other cardiac or cardiovascular patient.

In accordance with one aspect of the invention, stimulation to elicit a diaphragm response is provided to increase or normalize lung volume and in particular to increase functional residual capacity. It is believed that stimulation to increase or to normalize lung volume or functional residual capacity may have one or more effects that may be therapeutic to cardiovascular or heart failure patients. Normalizing herein may include for example, bringing a physiological parameter into a normal or healthy region for patients or for a particular patient, or to a level appropriate for a condition or state of a patient.

In accordance with another aspect of the invention stimulation is provided to control breathing to reduce respiration rate and thereby reduce hypertension, reduce sympathetic nerve bias, and/or provide improved blood gas levels.

In accordance with another aspect of the invention stimulation is provided to control minute ventilation to therapeutically effect blood gas levels.

In accordance with another aspect of the invention, stimulation is provided to create a deep inspiration or an increased tidal volume to thereby reduce sympathetic nerve bias, improve blood gas levels, stimulate reflexes for example the Hering-Bruer reflex related to activating stretch receptors, increase lung volume, normalize or reset breathing or provide other beneficial therapies to improve cardiovascular function or heart failure condition.

In accordance with another aspect of the invention stimulation may be provided to manipulate intrathoracic pressure to thereby produce a therapeutic effect. According to one embodiment, stimulation is provided to reduce intrathoracic pressure to thereby increase ventricular filling which may improve cardiac output.

In accordance with an aspect of the invention a combined cardiac rhythm management device and diaphragm/phrenic nerve stimulation device is provided to provide an enhanced combined treatment device.

A device in accordance with an aspect of the invention comprises a subcutaneously implanted diaphragm stimulator, i.e., configured to stimulate tissue to elicit a diaphragm response; and a subcutaneously positioned defibrillator.

A device in accordance with another aspect of the invention comprises a diaphragm stimulation device configured to operate in conjunction with a separately implanted subcutaneously positioned defibrillator

A diaphragm stimulator in accordance with one aspect of the invention may be configured to control or adjust stimulation in response to cardiac electrogram sensed by a cardiac electrogram sensor. The sensed cardiac electrogram may provide information on a cardiac condition or event and/or may control stimulation to avoid device-device interaction at the occurrence of a cardiac event. According to one variation, stimulation may be turned off when a cardiac event or condition is present for purposes of safety and to allow other intervention whether external or with implanted CRM device. The stimulator may also provide or adjust stimulation based on a cardiac event or condition, to therapeutically benefit the patient during such event or condition. An example of such sensor is set forth in co-pending application entitled: DIAPHRAGM STIMULATION DEVICE AND METHOD FOR USE WITH CARDIOVASCULAR OR HEART PATIENTS, filed on even date herewith and incorporated entirely herein by reference without limitation.

In accordance with another aspect of the invention, a diaphragm stimulation device is provided for treating disordered breathing and provides defibrillation to prevent sudden cardiac death. In accordance with one aspect of the invention, diaphragm stimulation leads are placed subcutaneously as described herein or directly on the diaphragm (as described in one or more related patent applications as set forth above and incorporated entirely herein by reference without limitation). The diaphragm stimulator includes electrocardiogram sensing that provides information on cardiac rhythm. The diaphragm stimulation device also includes a defibrillator configured to provide defibrillation signals when a ventricular defibrillation is detected.

These and other aspects of the invention are set forth herein in the abstract, specification and claims.

### DESCRIPTION OF THE DRAWINGS

FIG. 1 is a front schematic view illustrating a device in accordance with an aspect of the invention.

FIG. 2 is a front schematic view illustrating a device in accordance with an aspect of the invention.

### DETAILED DESCRIPTION

Examples of various devices for and uses of diaphragm stimulation are described in related applications set forth above and in copending patent application entitled: DEVICE AND METHOD FOR TREATING DISORDERS OF THE CARDIOVASCULAR SYSTEM OR HEART, filed on even date herewith, all of which are incorporated entirely herein by reference without limitation. A diaphragm stimulator in accordance with the invention may also include a cardiac electrogram sensor which may be used in conjunction with any diaphragm stimulation therapy.

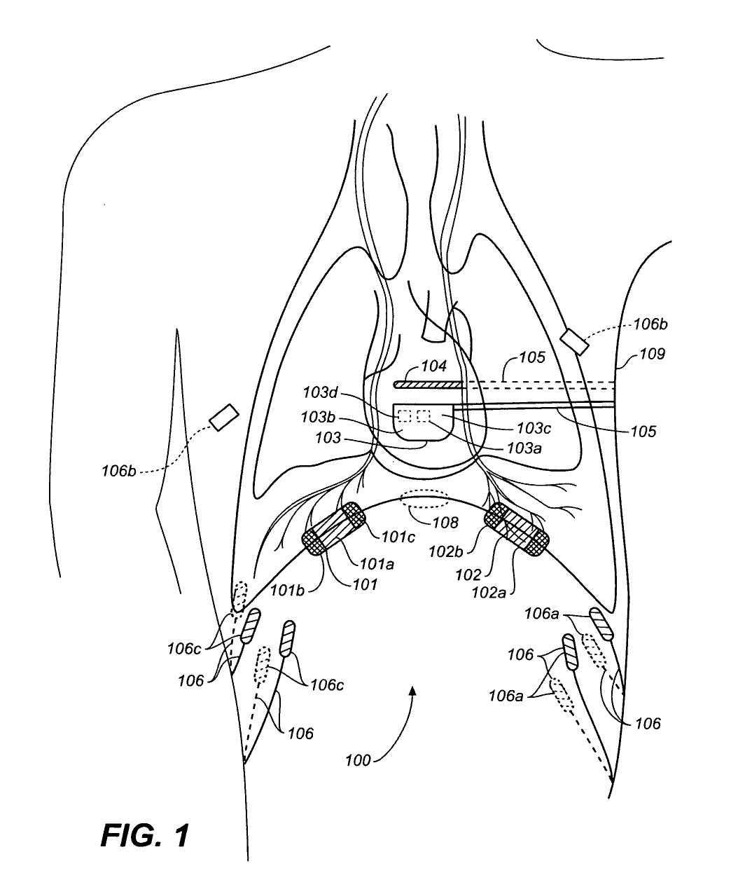

FIG. 1 illustrates a system 100 in accordance with the invention. The system 100 comprises a controller 103 configured to control both stimulation to elicit a diaphragm response and defibrillation. Accordingly controller includes a control circuit 103a configured to control diaphragm stimulation and defibrillation; a housing 103b having an electrically active portion 103c; and a capacitor discharge circuit 103d configured to transmit a discharged signal between the active portion 103c of the housing 103b to the anteriorly placed subcutaneous electrode (defibrillator) 104. Electrode assemblies 101 and 102 are positioned subcutaneously in the torso region on each side of the ziphoid process 108. In accordance with one aspect of the invention the electrode assemblies each comprise: a housing 101a and 102a which house electronic circuitry; and electrically active portions 101b, 101c and 102b, 102c respectively. The electrically active portions 101b and 101c may form bipoles for local stimulation of one hemidiaphragm while electrically active portions 102a and 102c may form bipoles for local stimulation of the other hemidiaphragm. One or more of the electrically active portions may be configured to sense diaphragm activity and/or cardiac activity. The electrode assemblies 101, 102 may include circuitry configured to receive a control and/or power communication signal from the controller (e.g., RF or other communication signal) to thereby supply electrical stimulation through electrically active portions 101b, 101c and/or 102b, 102c. The electrode assemblies 101, 102 may also be connected by leads to controller 103. Stimulation may be provided to the diaphragm, for example as set forth in one or more copending patent applications as set forth herein. Additional electrode assemblies 106 may be positioned to activate abdominal muscles (106a) or chest wall muscles (106b) during sleep apnea treatment. (Alternatively, electrode assemblies 101, 102 may be configured and positioned to individually activate diaphragm and abdominal muscles and/or chest wall muscles.) Such stimulation may be used to augment diaphragm stimulation (augment inspiration with chest wall stimulation or augment exhalation with abdominal muscle stimulation) and/or to synchronize stimulation of chest wall or abdominal muscles to avoid or reduce paradoxical abdominal or chest wall movement with diaphragm movement. The stimulation vectors of the diaphragm stimulator are arranged so that any electrode combination used to stimulate the diaphragm or other respiratory muscles do not stimulate the heart.

The system also includes a defibrillation electrode 104 located posterior to the controller 103 and coupled to the controller 103 by lead 105 subcutaneously positioned about the torso 109. A capacitor discharge may be applied from the discharge circuit 103d through the active portion 103c of the controller housing to the electrode 104. Other ICD electrodes as would be generally known may be used instead of one or more electrode assemblies or instead of defibrillation electrode 104. The controller 103 is configured to turn off noise detection and/or cancel a diaphragm stimulation signal that it controls, from detection algorithms that may be used to sense and detect noise. Thus diaphragm stimulation will not been seen as noise, the detection of which prevents defibrillation in some defibrillator algorithms. The subcutaneous defibrillation elements of system may be constructed and implanted as described, for example in U.S. Pat. No. 7,065,410 incorporated in its entirety herein by reference. One or more of the electrodes of the system may be used to sense an electrocardiogram and to determine when defibrillation is occurring.

In accordance with another aspect of the invention, one or more of the active portions 101b, 101c, 102b, 102c, or an additional active portion of housings 101a, 102a, of electrode assemblies 101, 102 may be used to provide a defibrillation vector.

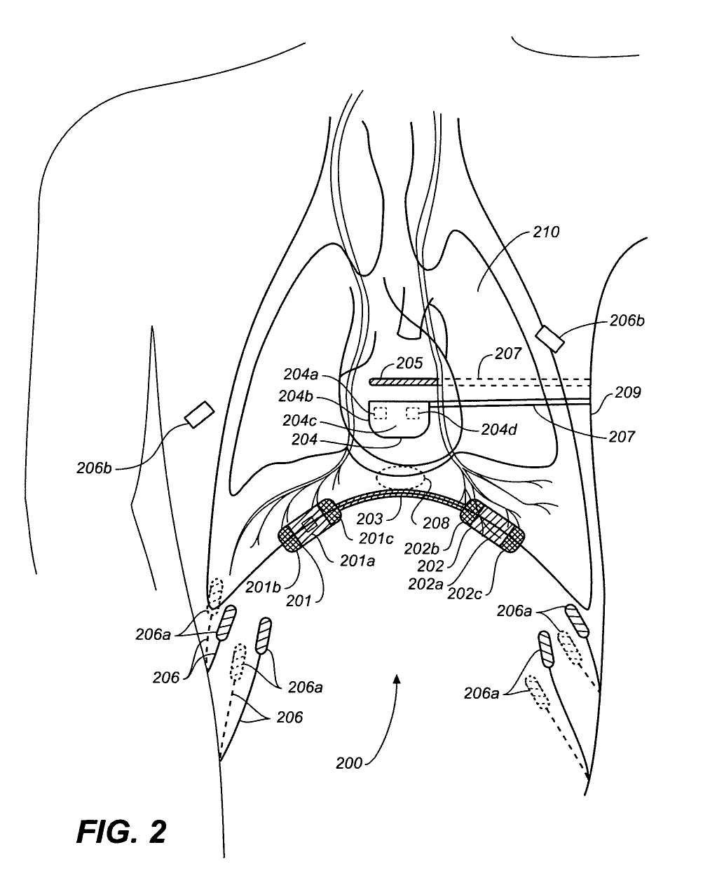

FIG. 2 illustrates a subcutaneously implanted diaphragm stimulation 200 with a separate controller 200a. The diaphragm stimulator 200 comprises electrode assemblies 201 and 202 positioned subcutaneously in the torso region on each side of the ziphoid process 208. The controller 200a is included with electrode assembly 201 which is coupled by way of lead 203 to electrode assembly 202. In accordance with one aspect of the invention the electrode assemblies 201, 202 each comprise: a housing 201a and 202a which house electronic circuitry; and having electrically active portions 201b, 201c and 202b, 202c respectively. The electrically active portions 201b and 201c may form bipoles for local stimulation of one hemidiaphragm while electrically active portions 202b and 202c may form bipoles for local stimulation of the other hemidiaphragm. Controller 200a controls electrically stimulating signals supplied to electrically active portions 201b, 201c and 202b, 202c of electrode assemblies 201 and 202 respectively. Stimulation may be provided to the diaphragm, for example as set forth in one or more copending patent applications as set forth herein. Additional electrode assemblies 206 may be positioned to activate abdominal muscles (206a) or chest wall muscles (206b) during sleep apnea treatment. (Alternatively, electrode assemblies 201, 202 may be configured and positioned to individually activate diaphragm and abdominal muscles and/or chest wall muscles.) Such stimulation may be used to augment diaphragm stimulation (augment inspiration with chest wall stimulation or augment exhalation with abdominal muscle stimulation) and/or to synchronize stimulation of chest wall or abdominal muscles to avoid or reduce paradoxical abdominal or chest wall movement with diaphragm movement.

The subcutaneous diaphragm stimulator 200 may also work with a defibrillator whether fully implanted or subcutaneously implanted. FIG. 2 illustrates a separate subcutaneous defibrillation system 210 comprising a controller 204, having a control circuit 204a, housing 204b with an electrically active portion 204c, and a capacitor discharge circuit 204d. The system also includes a defibrillation electrode 205 located posterior to the controller 204 and coupled to the controller 204 by lead 207 subcutaneously positioned about the torso 209. A capacitor discharge may be applied from the discharge circuit 204d through the active portion 204c of the controller housing to the defibrillation electrode 205. The subcutaneous defibrillation system may be constructed and implanted as described, for example in U.S. Pat. No. 7,065,410 incorporated entirely herein by reference. Other ICD electrodes as would be generally known may be instead of defibrillation electrode 205.

The stimulation vectors of the diaphragm stimulator are arranged so that any electrode combination used to stimulate the diaphragm or other respiratory muscles do not stimulate the heart.

The diaphragm stimulator 200 may be configured to avoid device-device interaction with defibrillation system 210. For example, defibrillation therapy produces a strong electric field in the body that may be sensed by at least one electrically active portions 201b, 201c, 202b, 202c or other sensor. One or more electrically active portions 201b, 201c on electrode assembly 201 may be arranged or selected in combination with one or more electrically active portions 202b, 202c on electrode assembly 202, for example, to provide a further reference such a global signal such as the defibrillation therapy would appear at the diaphragm stimulation device, is more effectively picked up. When the diaphragm stimulation device senses that defibrillation is occurring stimulation is adjusted or turned off to allow for defibrillation without interference from diaphragm stimulation.

According to another aspect of the invention, defibrillation system is configured to avoid negative device/device interaction with a diaphragm stimulator 200. Diaphragm stimulation produces a characteristic electric field in the body that may be sensed by a sensing electrode component of a defibrillation system 210, for example, based on a known stimulation signal characteristics such as frequency, amplitude and/or duration of the stimulation. The defibrillation system may be programmed to recognize one or more of these characteristics. Programming and detection may occur, for example, when the diaphragm stimulation device is implanted. The electrode 205 and/or electrically active portion 204d may be arranged, to picks up a global signal such as the diaphragm stimulation. If the defibrillation system 210 senses that diaphragm stimulation is occurring then it may be configured to adjust it's sense algorithms for reduced sensitivity to a diaphragm stimulation artifact.

The defibrillation system and diaphragm stimulation device may be combined into a single device and/or a device with a single implanted control unit.

1. A cardiovascular treatment assembly comprising:
a cardiac rhythm management device; and
an electrical stimulation device configured to elicit a diaphragm contraction to thereby treat heart failure.

2. The assembly of claim 1 where the cardiac rhythm management device comprises a subcutaneously implantable defibrillator.

3. The assembly of claim 1 wherein the electrical stimulation device at least one electrode selected from a group comprising: electrodes implanted at the diaphragm, electrodes implanted on the phrenic nerve, an electrode implanted transvenously, and electrode located externally and an electrode located subcutaneously.

4. The cardiovascular treatment assembly of claim 1 wherein the electrical stimulation device is configured to reduce breathing rate.

5. The cardiovascular treatment assembly of claim 1 wherein the electrical stimulation device is configured to increase functional residual capacity.

6. A device comprising
a subcutaneously implantable defibrillator; and
an electrical stimulation device configured to elicit a diaphragm contraction;
a controller configured to coordinate the subcutaneous defibrillator and the electrical stimulation device.
